(19) United States
(12) Patent Application Publication
(10) Pub. No.: US 2025/0111535 A1
(43) Pub. Date: Apr. 3, 2025

(54) METHOD AND MEASURING DEVICE FOR CORRECTING A POSITION OF A MEASUREMENT POINT

(71) Applicant: KARL STORZ SE & Co. KG, Tuttlingen (DE)

(72) Inventors: Simon HAAG, Tuttlingen (DE); Jasmin KEUSER, Tuttlingen (DE); Lukas BUSCHLE, Tuttlingen (DE)

(73) Assignee: KARL STORZ SE & Co. KG, Tuttlingen (DE)

(21) Appl. No.: 18/832,371

(22) PCT Filed: Jan. 23, 2023
(86) PCT No.: PCT/EP2023/051513

§ 371 Date: Jul. 23, 2024

(30) Foreign Application Priority Data
Jan. 24, 2022 (DE) 10 2022 101 524.6

(51) Int. Cl. G06T 7/73; A61B 1/00

(52) U.S. Cl. CPC G06T 7/74; A61B 1/000095; G06T 2207/10021; G06T 2207/10056; G06T 2207/10068

(57) ABSTRACT

A method for correcting a position of a measurement point in an endoscopic and/or exoscopic and/or microscopic measurement image, at least comprising the steps of: capturing a first measurement image of at least one movable measurement object; determining the at least one measurement point in relation to the at least one movable measurement object in the first measurement image; capturing at least one second measurement image, following the first measurement image in time, of the at least one movable measurement object; calculating a position displacement vector between the at least one measurement point in the first measurement image and an image point, corresponding to the at least one measurement point, in the second measurement image; and correcting a position of the at least one measurement point determined in relation to the first measurement image, in the second measurement image on the basis of the calculated position displacement vector.

### CROSS-REFERENCE TO RELATED APPLICATIONS

This application is the U.S. national stage of PCT/EP2023/051513 filed on Jan. 23, 2023, which claims priority of German Patent Application No. 10 2022 101 524.6 filed on Jan. 24, 2022, the contents of which are incorporated herein.

### TECHNICAL FIELD

The present disclosure relates to a method for correcting a position of a measurement point in a measurement image, in particular in an endoscopic and/or exoscopic and/or microscopic and/or laryngoscopic measurement image. The disclosure also relates to a method for outputting at least one statistical characteristic variable for a movable measurement object, which is in particular comprised in an endoscopic and/or exoscopic and/or microscopic and/or laryngoscopic measurement image. Furthermore, the disclosure relates to a measuring device with a processor designed to perform at least one of the methods according to the disclosure.

### BACKGROUND

Optical visualization systems, such as microscopes, exoscopes and endoscopes, make it possible to represent a scene and/or work area in which fine motor work and/or visual examinations are carried out. In medical procedures, the work area is, for example, an operating field in an internal area, for example within a thorax or a head, of the human body.

Exoscopy describes observation and, if necessary, illumination of an operating field on a patient and/or of an object field on any object from a location away from, i.e., outside, the patient's body or away from the object.

Endoscopy describes an imaging technique in which an endoscope is inserted into a cavity. The specialists who carry out such a procedure with an endoscope view the image, captured by the endoscope, on the screen and can direct their actions on the basis of this image. In medical, for example minimally invasive, procedures, an endoscope is inserted into the body in order to capture an internal image of the body and display it on the screen. Due to the often required precise way of working of the specialists, it is desirable to provide as accurate, high-resolution an image as possible of the cavity and/or work area where the examination and/or operation is to be carried out.

The images captured by the endoscope and displayed on a display device are generally two-dimensional so that, due to the lack of depth information, even the specialists are unable to exactly determine dimensions and/or measurements of a viewed object in the displayed scene.

However, in order to solve this problem and/or to also make depth measurements in a measurement image possible, three-dimensional stereo endoscopes and/or stereo exoscopes and/or microscopes are also known, in which a scene is recorded from two different viewing angles by two image capture units. The two image capture units each record an image of the scene to be viewed, preferably in a manner synchronized per time unit and viewing angle. This in each case results in so-called stereo measurement image pairs with a common time stamp. Depth information can be evaluated from such a stereo measurement image pair via stereo reconstruction methods.

As part of this stereo reconstruction, a disparity, i.e., a horizontal pixel offset, in each of the stereo image pairs is determined pixel by pixel, i.e., per pixel, by an algorithm. In stereo capture, objects at a great distance exhibit a small disparity, i.e., a small pixel offset, within a particular image pair. In contrast, objects in the foreground exhibit a large disparity.

From the disparity, with knowledge of further optical parameters of the image capture unit(s), a type of depth map can be calculated for the stereo-reconstructed stereo measurement image, which map, for example, comprises pixel-by-pixel depth information of the object to be viewed. By means of the depth information, it is possible, for example, to determine a distance between any two image points in Euclidean space.

The prior art already discloses methods and devices that make dimensional measurements possible between two measurement points in Euclidean space and thus on a measurement object by using depth information of the particular measurement points. Here, a user selects, for example via a manual input, two measurement points on a measurement object in a stereo measurement image, between which a distance is to be calculated. This makes it possible for the user to select points within the stereo measurement image, for example, and, subsequently thereto, for a distance between these points to be output, for example as a superimposed representation in the displayed stereo measurement image.

WO2021/138262 A1 and EP1965699 B1 each disclose a medical robot system in which, in general, a superimposed representation of information in measurement images takes place by means of “telestration.” Telestration describes the commenting and marking of images in the operating room by a mentor or another person who attends the operation externally, i.e., for example, via live video. In this case, it is possible to represent telestration graphics in a captured stereo image and, for example, to assign them to a specific measurement object in the stereo image.

It is also known from WO 2019/213432 A1 that a user can select measurement points on a measurement object in a two-dimensionally represented measurement image, which measurement points are subsequently converted into three-dimensional measurement points by assigning disparity-based depth information. By assigning their respective depth information, it is possible for the user to set measurement points along a contour of the measurement object with only a two-dimensional representation of the measurement object in relation thereto in such a way that, for example, a dimensional measurement along the contour is made possible.

The prior art also discloses a solution approach in which the user can select measurement points on a measurement object with a stereo endoscope in real time during a video recording in order to subsequently have a distance between these measurement points displayed as a superimposed representation in the endoscopic image. In order to compensate for possible movements of the image capture unit(s), the movement of the image capture unit(s) is detected by sensors and the selected measurement points are tracked optically in the stereo measurement image. The measurement points in the measurement image can be adjusted on the basis of the movement data from the sensors so that any movement of the image capture unit(s) is compensated.

Although the depth-information-based selection of measurement points on a measurement object is known from the prior art, the methods and systems are inadequate if the measurement object moves during the observation. For example, in the medical field, when capturing moving measurement objects, in particular organs, for example, the lungs, heart, intestine, etc., the measurement object moves back and forth while a measurement point is being selected, which makes reliable selection and/or targeting with a cursor difficult or even impossible. This makes it difficult for the user to correctly set a measurement point in relation to the measurement object.

In the case of measurement objects that move, in particular rhythmically and/or with a frequency, the observer and/or user is likewise faced with the challenge that a measuring section between at least two measurement points that have been set in relation to the measurement object by the observer is subject to temporal changes due to the movement of the measurement object. For example, a measurement value for the measuring section in a snapshot may not be displayed correctly since the measurement object has already moved relative to its previous position and an actual distance in the viewed snapshot has thus become either smaller or larger. The measurement accuracy of known dimensional measuring methods and measuring systems is thus insufficient and/or an accuracy achievable therewith is regularly overestimated by the user.

### SUMMARY

The present disclosure is therefore based on an object of overcoming the aforementioned disadvantages of the prior art. In particular, it is an object to provide a method and a measuring device by means of which an accuracy in determining a position of a measurement point on a moving measurement object is increased and/or made easier for a user. In particular, it is also an object to specify a method and a measuring device by means of which a measured distance and/or a measurement accuracy in a dimensional measurement between two measurement points on a measurement object can be determined as precisely as possible, even in the case of a moving measurement object, so that information that is as simple as possible but nevertheless meaningful can be shown and/or displayed to the user in an, in particular endoscopic, measurement image.

At least one of the aforementioned objects is achieved by a method with the features of independent claim 1. An alternative or supplementary solution to at least one of the aforementioned objects is provided by a method with the features of independent claim 8. Furthermore, the objects are achieved by a measuring device according to the disclosure which is designed to carry out at least one of the methods according to the disclosure.

Advantageous developments of the disclosure are specified in the dependent claims. The scope of the disclosure includes all combinations of at least two of the features disclosed in the description, the claims and/or the figures. It is understood that exemplary embodiments and embodiments that are described with respect to the method according to claim 1 may relate in an equivalent, albeit not equally worded, form to the method according to claim 8 without being explicitly mentioned therefor. It is also understood that customary linguistic transformations and/or a corresponding replacement of particular terms within the framework of customary linguistic practice, in particular the use of synonyms supported by generally accepted linguistic literature, are also included in the present disclosure content without being explicitly mentioned in their particular formulation.

According to a first aspect of the disclosure, a method for correcting, in particular graphically, a position of a measurement point in a measurement image, in particular in an endoscopic and/or exoscopic and/or microscopic and/or laryngoscopic measurement image, is specified. The method comprises at least the following steps: capturing a first measurement image of at least one movable measurement object; determining the at least one measurement point in relation to the at least one movable measurement object in the first measurement image; capturing at least one second measurement image, following the first measurement image in time, of the at least one movable measurement object; calculating a position displacement vector between the at least one measurement point in the first measurement image and an image point, corresponding to the at least one measurement point, in the second measurement image; and, on the basis of the calculated position displacement vector, correcting a position of the at least one measurement point set in relation to the first measurement image, in the second measurement image.

Particularly preferably, the first and/or second measurement image is in each case an already stereo-reconstructed stereo measurement image, in which depth information is preferably made available in each case at least for each pixel in relation to the at least one movable measurement object on the basis of a performed stereo reconstruction. In order to capture the first stereo measurement image, one measurement image of the movable measurement object is preferably recorded from each of two different viewing angles (predetermined by a stereo basis between a first and a second image capture unit) in a synchronized manner with regard to a predetermined time unit. The two measurement images, which are each captured synchronously in time, preferably form a stereo measurement image pair with a common time stamp. From each stereo measurement image pair, depth information can be evaluated pixel by pixel per time stamp by means of stereo reconstruction methods. Preferably, as part of the stereo reconstruction, a so-called disparity, i.e., a horizontal pixel offset, between each measurement image of the respective stereo image pair is determined pixel by pixel, i.e., per pixel, by an algorithm. In stereo capture, objects at a great distance preferably exhibit a small disparity, i.e., a small pixel offset, within a particular stereo image pair. In contrast, objects in the foreground exhibit a large disparity. From the disparity ascertained pixel by pixel, with knowledge of further optical parameters of the image capture unit(s), a depth map can preferably be calculated for the stereo-reconstructed first and/or second stereo measurement image, which map preferably comprises pixel-by-pixel depth information of the object to be viewed. By means of the depth information, it is possible, for example, to determine a distance between any two measurement points within the first and second stereo measurement image in Euclidean space.

Alternatively, the first and second measurement images can each also be a measurement image in which, in particular pixel-by-pixel, depth information, i.e., a respective distance to points of an object, preferably to each pixel, is ascertained with a so-called time-of-flight sensor. Endoscopes with such sensors are known from the prior art, for example from EP 2 599 433 B1.

Alternatively, the first and second measurement images can each also be a measurement image in which, in particular pixel-by-pixel, depth information is determined in each case on the basis of a so-called pseudo-stereoscopy. In this pseudo-stereoscopy, the depth information is obtained from a stereo measurement image that comprises two temporally spaced individual images or frames of a video of a captured movement of a measurement object. In this case, the first and second measurement images are thus each a stereo measurement image. The individual images are preferably recorded by a conventional (mono) optical system, preferably overlap and can be used similarly to a stereo measurement image pair or similarly to the measurement images of a first and second stereo channel. The, in particular pixel-by-pixel, depth information for each measurement image can be ascertained by means of stereo reconstruction methods.

Alternatively, the first and second measurement images can also be measurement images in which the, in particular pixel-by-pixel, depth information is ascertained on the basis of artificial intelligence. For example, it is possible to ascertain 3D information from 2D images on the basis of artificial intelligence. Such measurement images can be captured, for example, by a (mono) optical system or (mono) camera. Three-dimensional image information, i.e., depth information, can then be deduced from the captured two-dimensional measurement images by applying an algorithm that, for example, represents an artificial neural network.

Alternatively, the first and second measurement images can each also be measurement images in which, in particular pixel-by-pixel, depth information is ascertained in each case on the basis of size ratios of previously known structures in the measurement scene. These structures can, for example, be a part of an instrument and/or a marking on an instrument that is recognizable in the respective captured measurement image in order to estimate a distance of the camera from the structure on the basis of a two-dimensional representation of the structure in the measurement image. On the basis of this estimate, the respective distance of further image points in the measurement image can preferably be deduced.

According to the disclosure, the correction of the position change of the at least one measurement point is preferably used to make it easier for the user to select measurement points in a measurement image which shows a moving measurement object. The tracking according to the disclosure of a position change at least between the first and the second measurement image preferably takes place by mathematical-optical tracking (optical tracking).

In the present case, the first and the second measurement image are each individual images of a plurality of temporally successive measurement images which, when placed one after the other, result in a video recording of at least one measurement object. A time interval between the individual images is preferably determined by a predetermined image capture frequency (frame rate). It is understood that the first measurement image can in principle be any individual image of such a video recording and/or video sequence and of course does not have to be an initially captured image. It is also understood that the second measurement image in principle does not necessarily have to follow the first measurement image immediately in terms of time, but that one or more measurement images can be captured in between. The position of the at least one measurement point determined in relation to the first measurement image is preferably corrected in the second measurement image on the basis of the calculated position displacement vector in real time so that the user preferably cannot visually detect this position displacement and it appears to them as if the at least one measurement point adheres to the at least one measurement object.

In other words, the at least one measurement point is preferably tracked, preferably continuously, between successive individual images (frames) of a live stereo video recorded of the at least one measurement object. This makes it possible, for example, to correct a position of a cursor, which is moved by a user at least through the first measurement image in order to determine and/or set and/or select the at least one measurement point, in the second measurement image. More generally, it is possible to correct the position change of the at least one measurement point in each second measurement image that follows a first measurement image in time. Thus, it is possible to compensate for the movement of the at least one measurement object by adjusting the measurement point in each case. From the perspective of the user, the at least one measurement point, for example represented as a cursor, thus sticks to the at least one measurement object. If the user wants to move the cursor, i.e., the measurement point to be set in relation to the measurement object, this movement is superposed according to the disclosure with the calculated position displacement resulting from the movement of the measurement object. In other words, the user displaces the cursor relative to the moving measurement object and not a pixel position of the cursor on a screen.

As an alternative to the solution according to the disclosure, it would be possible to select the at least one measurement point on a still image. However, this would have the disadvantage that no temporal information about the distance of the measurement point from the image capture device would be available. This would result in the measurement result being inaccurate in terms of time. In addition, it would, for example, be cumbersome for the user to add an additional window to the workflow.

It is understood that the method steps according to the disclosure do not necessarily have to be carried out in the order listed, but that this order can also be changed. It is also possible that one or more intermediate steps can be carried out between one or more method steps.

In a preferred embodiment, the method according to the disclosure according to the first aspect is characterized in that a position change between the at least one measurement point determined in relation to the first measurement image and the image point corresponding thereto in the second measurement image is based on an, in particular rhythmic and/or periodic and/or frequency-based, movement of the movable measurement object. The method according to the disclosure thus differs from the prior art in particular in that the position of a measurement point in relation to the measurement object is corrected not only on the basis of a change in the location and/or position of the measuring device. Instead, the position of the measurement point is corrected on the basis of the movement of the measurement object so that the measurement point appears to visually adhere to the measurement object. This has previously not been possible in the prior art. The position displacement vector is thus preferably calculated on the basis of the movement of the at least one measurement object. The rhythmic and/or periodic and/or frequency-based movement of the movable measurement object results in the measurement object continuously moving relative to the measuring device used according to the method, in particular relative to the two image capture units, and thereby, inter alia, continuously changing its distance to each image capture unit. As a result, the depth information for the individual image points in relation to the measurement object also changes continuously between two measurement images. If a user determines a measurement point in relation to the measurement object, this determination generally takes place on the basis of specific depth information. According to the disclosure, in particular by calculating the position displacement vector, it is now possible to take into account the distance information and/or depth information, which changes over time due to the movement of the measurement object, which can take place in all three spatial directions, in the determination of the measurement point. As a result, the measurement point adheres to at least one measurement object.

In a further preferred embodiment, the method according to the disclosure according to the first aspect is characterized in that calculating the position displacement vector takes place on the basis of an optical flow algorithm and/or an elastic image registration algorithm and/or a point cloud registration algorithm and/or landmark-based tracking. In principle, it is understood that the algorithmic calculation methods listed here are not to be understood as restrictive, but that other and/or supplementary calculation methods can also be used to calculate the position displacement vector according to the disclosure.

In a further preferred embodiment, the method according to the disclosure according to the first aspect is characterized in that the first and the second measurement image are each an individual image of a video sequence. Preferably, the at least one movable measurement object is continuously captured as a video recording, in particular by an endoscopic and/or exoscopic and/or microscopic and/or laryngoscopic measuring device. Preferably, a first and a second image capture unit respectively make such a video recording of the at least one movable measurement object. The first image capture unit preferably records the measurement object from a first viewing angle. The second image capture unit preferably records the measurement object from a second viewing angle, wherein the first viewing angle differs from the second viewing angle. For the video recording of the measurement object, the first image capture unit preferably generates a plurality of individual images (frames) that are temporally successive according to a predetermined image capture rate (frame rate, measured in frames per second). For the video recording of the measurement object, the second image capture unit preferably generates a plurality of individual images (frames) that are temporally successive according to a predetermined image capture rate (frame rate, measured in frames per second). The image capture rate of the first image capture unit preferably corresponds to the image capture rate of the second image capture unit so that the two image capture units record individual images that are temporally synchronized with one another.

In a further preferred embodiment, the method according to the disclosure according to the first aspect is characterized in that determining the at least one measurement point in relation to the at least one movable measurement object takes place on the basis of a user input, which is preferably made by moving a cursor on a display within the first measurement image. The user input may preferably comprise a manual user input or a semi-automatic or automatic user input. For example, the user input may take place manually by means of a joystick, keyboard, mouse, and/or touch screen. Alternatively or additionally, the user input may also be partially automated, for example computer-assisted. Automatic user input, particularly carried out by a computer, is also possible.

In a further preferred embodiment, the method according to the disclosure according to the first aspect is characterized in that correcting the position of the at least one measurement point comprises superimposing a movement vector of the cursor for determining the measurement point with the position displacement vector. According to this exemplary embodiment, it is possible for the cursor to be moved to a targeted measurement point in the measurement image, for example during a live video capture of the measurement object. Preferably, during such a movement within the measurement image, the cursor is already continuously corrected on the basis of the position displacement vector, which is preferably dependent on a periodic and/or frequency-based movement of the measurement object.

In a further preferred embodiment, the method according to the disclosure according to the first aspect is characterized in that the at least one measurement point is displayed on a user output device, preferably always and/or continuously and/or without interruption, in its corrected position. The position correction as such is thus preferably not visualized to the user. As a result, the user only notices that the at least one measurement point adheres to at least one movable measurement object, i.e., always follows it during its movement. The at least one measurement point thus preferably moves back and forth with the at least one measurement object in the (live) video recording in front of the user's eye. This makes it possible for the user to set the measurement point with greater accuracy in relation to the at least one measurement object, since the user can, for example, track a desired location at which the measurement point is to be set, even during the movement of the measurement object and can thus ensure that the measurement point has been correctly placed in relation to the measurement object.

According to a preferred embodiment, it is possible for a movement of the image capture device preferably used for the method according to the disclosure to be compensated. For this purpose, inter alia, a position change of the at least one measurement point between the first and the second measurement image, which change can, for example, be caused by the movement of the image capture device and/or by a movement of the at least one movable measurement object, is tracked by, in particular optical, tracking methods. Preferably, during optical tracking, it is not the at least one measurement point that is tracked, but the observed measurement object as such. For such tracking of the movement of the image capture device itself, a larger number of recognized (measurement) points, e.g., at particularly prominent structures such as edges or high-contrast areas of the measurement object, is preferably known. Preferably, at least two measurement points on the measurement object are known, particularly preferably a plurality of measurement points. In this way, a translational movement in space and/or a rotation of the image capture device can preferably be derived from the tracked measurement points. It is understood that other types of tracking may also be used. As a result, a modified position displacement vector can preferably be calculated, through which a movement of the image capture device can also be taken into account. Preferably, the image capture device has at least one position sensor, for example a gyro sensor, a GPS sensor and/or an optical sensor, by means of which a position change of the image capture device can be detected in the form of a sensor signal. The image capture device may also comprise a medical navigation system configured to optically or electromagnetically detect a position of the image capture device from outside the image capture device. For this purpose, the image capture device may, for example, comprise a tracker or a magnetic field sensor in conjunction with a coil for electromagnetic detection. Such a sensor signal is preferably included in the calculation of the modified position displacement vector. This makes it possible to correct the position change of the at least one measurement point at least in the second measurement image so that the at least one measurement point in the user-related representation appears to adhere to the at least one movable measurement object, i.e., preferably does not move relative thereto. A further advantage of the at least one measurement point being adjusted and/or its position being corrected is that any measurement values and/or other information selected by the user for this measurement point (e.g., a preferably three-dimensional position of the measurement point in a predetermined coordinate system) can be averaged over time, in particular over a plurality of measurement images. Measurement errors can thus be compensated.

In a second aspect of the disclosure, a method for outputting at least one statistical characteristic variable for a movable measurement object, which is in particular comprised in an endoscopic and/or exoscopic and/or microscopic measurement image, is specified. The method according to the disclosure comprises at least the steps described below. The method comprises providing a plurality of temporally successive measurement images of the at least one movable measurement object by an image capture device, wherein the plurality of measurement images each comprise, in particular, information about at least one measuring section to be output in relation to the movable measurement object. The measuring section is calculated between a first measurement point, which is determined in relation to the at least one movable measurement object, and a second measurement point, which is determined in relation to the at least one movable measurement object. The measuring section preferably defines a distance to be measured between the first and the second measurement point. Furthermore, the method comprises ascertaining, by statistical evaluation, the at least one statistical characteristic variable, in particular with respect to the at least one measuring section and/or the distance between the first and the second measurement point, in the plurality of temporally successive measurement images, which statistical characteristic variable is at least partially related to a rhythmic and/or periodic and/or frequency-based movement of the at least one movable measurement object. The method furthermore comprises outputting the ascertained, at least one statistical characteristic variable, which describes the movement of the at least one movable measurement object at least partially, preferably in its entirety. The at least one statistical characteristic variable is preferably output in graphical and/or textual and/or acoustic and/or tactile form to a user of the method.

Instead of the static examination according to the disclosure of the plurality of temporally successive measurement images or the time series of at least one measuring section on periodic structures, it would also be possible as an alternative solution to display the measurement results of each measurement image to the user. However, in comparison to the solution according to the disclosure, this solution approach would have the disadvantage that the displayed measured distances would change quickly or jump back and forth between the individual measurement images. This would make it difficult for the user to read the measurement values precisely.

Thus, at least one of the aforementioned objects underlying the disclosure is achieved in that, preferably for each measuring section, a plurality of measurement results is determined from temporally successive measurement images on the basis of stereo reconstruction methods. Due to the plurality of measurement results, it is in particular possible to represent an effect of a movement of the at least one movable measurement object on the at least one measuring section. The first and/or the second measurement point are preferably set in relation to real points of the at least one measurement object and, if necessary, its surroundings. If at least one of these real points moves due to a movement of the measurement object, the, in particular Euclidean, distance between the two measurement points changes depending on the type and extent of the movement. The user can thus select the at least two measurement points, preferably in real time. The method according to the disclosure preferably displays to the user a distance between the two selected measurement points, preferably as a graphic superimposition in a measurement image, particularly preferably as a graphic superimposition in a stereo video recording of the at least one measurement object.

In a present method, a plurality of measurement results for a distance to be measured between two measurement points on a measurement object, in particular a time series of measurement results, can be ascertained on the basis of stereo reconstruction of measurement images. This plurality of measurement results is evaluated, for example by averaging, in order thus to obtain a time-averaged distance between two measurement points.

According to the disclosure, a movement of the measurement object and thus a temporal change in the distance is detected through stereo reconstruction of a plurality of temporally successive measurement images. The detected time profile of the distance, in particular a time profile of a change in distance, is statistically evaluated according to the disclosure in such a way that at least one statistical characteristic variable that is characteristic of the measurement object movement can be detected in the plurality of temporally successive measurement images and can be output to the user. In this way, it is, for example, possible to determine a periodicity, in particular a frequency and/or an amplitude, of the measured distance. According to the disclosure, it is thus possible to output to the user, preferably in addition to a static average value, at least one statistical characteristic variable, for example a minimum value and/or a maximum value and/or a movement frequency, for the measuring section. It should be mentioned that the at least one statistical characteristic variable may also be an average value formed in terms of time. The inventors also recognized that this averaging can lead to insufficient measurement accuracy.

The solution to this problem is the statistical evaluation and analysis of the time series of the at least one measuring section. In this case, statistical evaluation functions by means of which a movement of the at least one measurement object can be described as precisely as possible are preferably applied to the time profile of the measuring section. The statistical evaluation thus preferably also makes it possible to trace changes in a frequency and/or a rhythm and/or a period of the movement, so that an accurate description of the movement can always be output to the user, even in the case of complex movement sequences.

In a preferred embodiment, the method according to the disclosure according to the second aspect is characterized in that the at least one statistical characteristic variable comprises at least one of a frequency, an amplitude, a minimum value, a maximum value, a statistical average value, a standard deviation, and a statistical error indicator. It may also comprise other statistical values that are not explicitly mentioned in the above list. The selection of the output of the at least one statistical characteristic variable is preferably dependent on the movement of the at least one movable measurement object. Preferably, a plurality of statistical characteristic variables is output to the user so that the movement of the measurement object is described as accurately as possible. Particularly preferably, the statistical evaluation ascertains a time profile of the at least one statistical characteristic variable.

In a preferred embodiment, the method according to the disclosure according to the second aspect is characterized in that an average resulting measurement error is output for the at least one statistical characteristic variable. By outputting the measurement error, the user is able to recognize whether a currently performed distance measurement still meets the desired accuracy requirements or whether, for example, a movement of the at least one movable measurement object has changed in such a way that a distance measurement cannot currently take place with the required accuracy. The output of the measurement error comprises a warning function for the user, which warning function can be output to the user, for example, as an acoustic, graphical, visual and/or haptic indication.

In a preferred embodiment, the method according to the disclosure according to the second aspect is characterized in that a position change of the first measurement point and/or of the second measurement point in the plurality of temporally successive measurement images, which position change is caused at least partially by a movement of the at least one movable measurement object, is corrected by the method according to the disclosure according to the first aspect of the disclosure and according to the preferred embodiments thereof. The position of the two measurement points, which were preferably determined in relation to the at least one measurement object by the user for defining the at least one measuring section, can thus be corrected by the method according to the disclosure according to the first aspect in such a way, preferably in real time, that at least one of the two measurement points visually appears to adhere to the at least one measurement object. It is possible that the position of only one of the two measurement points is corrected relative to the at least one measurement object by the method according to the disclosure.

In a preferred embodiment, the method according to the disclosure according to the second aspect is characterized in that the first measurement image and/or the second measurement image and/or the plurality of measurement images are processed by stereo reconstruction of a respective stereo image pair on the basis of optical and/or dimensional parameters of the image capture device. In this embodiment, the first and the second measurement image are preferably each a stereo measurement image, each formed from a stereo measurement image pair. The processing preferably comprises an, in particular pixel-by-pixel, calculation of depth information for each image point in the first and/or the second measurement image (stereo reconstruction) in order thus to make possible a dimensional measurement between at least two measurement points selected from a recorded scene. The stereo reconstruction preferably comprises a correction of distortion effects and/or a transformation in each stereo image pair (rectification).

According to a third aspect of the disclosure, a measuring device is specified. The measuring device comprises an image capture device with at least a first and a second image capture unit spaced apart from the first one, and at least one evaluation device. It is understood that the evaluation device can be arranged outside or inside the image capture device and is configured to interact with the image capture device. Furthermore, the measuring device comprises a processor, in particular an evaluation and/or computing unit. The processor can preferably be comprised in the evaluation device or arranged separately therefrom. The processor is preferably configured to at least partially perform the steps of the method according to the first aspect of the disclosure, including embodiments thereof. Alternatively or additionally, the processor is configured to at least partially perform the steps of the method according to the second aspect of the disclosure, including embodiments thereof.

According to the third aspect, the processor is preferably configured to capture and/or ascertain a first measurement image, preferably in the form of measurement image information, of at least one movable measurement object. Furthermore, the processor can be configured to provide the computing power that is technically and graphically necessary for determining the at least one measurement point in relation to the at least one movable measurement object in the first measurement image. The processor is preferably configured to process at least one second measurement image of the at least one movable measurement object, which second measurement image follows the first measurement image in time. Furthermore, the processor is configured to calculate a position displacement vector between the at least one measurement point in the first measurement image and an image point, corresponding to the at least one measurement point, in the second measurement image and to correct a position of the at least one measurement point, set in relation to the first measurement image, in the second measurement image on the basis of the calculated position displacement vector.

Alternatively or additionally, according to the third aspect, the processor can preferably be configured to provide a plurality of temporally successive measurement images of the at least one movable measurement object. The image capture device is preferably configured to transmit a plurality of temporally successive stereo image pairs in the form of stereo image pair information to the processor. The processor is preferably configured to generate or provide a measurement image in each case from each measurement image pair by stereo reconstruction. The plurality of measurement images comprise at least one measuring section which is to be output in relation to the movable measurement object and which is determined between a first measurement point, which is determined in relation to the at least one movable measurement object, and a second measurement point, which is determined in relation to the at least one movable measurement object. The processor is preferably designed to calculate a distance of the at least one measuring section. Furthermore, the processor is configured to ascertain, by statistical evaluation, at least one statistical characteristic variable in the plurality of temporally successive measurement images, which statistical characteristic variable is at least partially related to a periodic and/or frequency-based movement of the at least one movable measurement object. The processor is preferably configured to transmit the at least one statistical characteristic variable or a time profile of the at least one statistical characteristic variable to the user output device. The user output device is configured to output the at least one statistical characteristic variable, which at least partially describes the movement of the at least one movable measurement object in terms of time.

In a preferred embodiment, the measuring device comprises a stereo endoscope and/or a stereo exoscope and/or a stereo microscope and/or a laryngoscope. Preferably, the measuring device is designed as a stereo endoscope and/or as a stereo exoscope and/or as a stereo microscope and/or as a laryngoscope.

In a preferred embodiment, the measuring device comprises a user input device and/or a user output device. The user input device may comprise a keyboard and/or a mouse and/or a joystick and/or a touch screen and/or a touch pad and/or another manual input device. The user output device may comprise a screen and/or glasses and/or 3D glasses and/or augmented-reality glasses.

Further advantages and details of the disclosure become apparent from the following description of preferred embodiments of the disclosure and from purely schematic drawings.

### BRIEF DESCRIPTION OF DRAWINGS

FIG. 1: is a schematic view of an exemplary embodiment of a measuring device according to the disclosure;

FIG. 2: is an exemplary stereo measurement image in which an exemplary movable measurement object is captured in a first position, with a measured distance represented in relation to the measurement object;

FIG. 3: is an exemplary stereo measurement image in which an exemplary movable measurement object is captured in a second position, with a measured distance represented in relation to the measurement object;

FIG. 4: is a graphical representation of a time profile of a measured distance;

FIG. 5: is the graphical profile, shown in FIG. 4, of the measured distance with a superimposed representation of a time profile of an average value of the measured distance;

FIG. 6: is an exemplary frequency diagram captured by a statistical evaluation;

FIG. 7: is the graphical profile, shown in FIG. 4, of the measured distance with a superimposed representation of statistically calculated minima and maxima.

FIG. 8: is an exemplary arrow representation of a cursor for an exemplary representation of a position displacement vector and a movement vector of the cursor, which occurs due to a user input, between successive stereo measurement images.

### DETAILED DESCRIPTION OF PREFERRED EMBODIMENTS

Identical elements or elements with the same function are provided with the same reference signs in the figures.

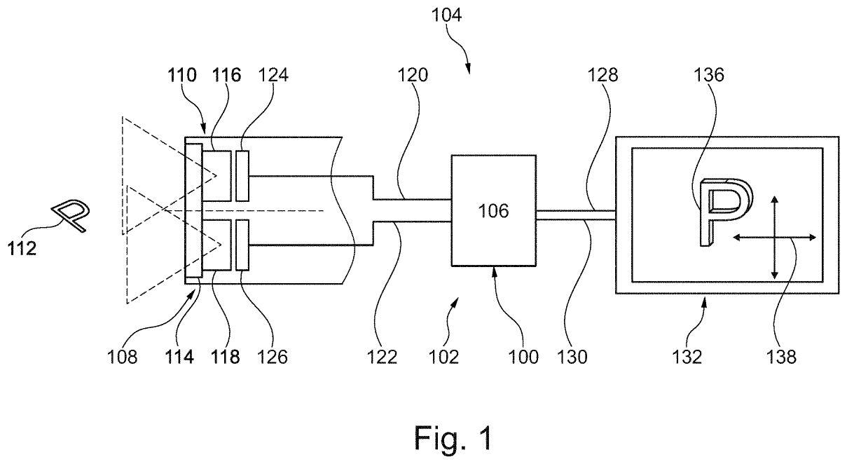

FIG. 1 shows an exemplary configuration of a measuring device 100 in a schematically greatly simplified block diagram. The measuring device 100 here is designed as a stereo endoscope 102. The measuring device 100 comprises an image capture device 104 with an evaluation device 106. Furthermore, the image capture device 104 comprises a first image capture unit 108 and a second image capture unit 110.

The first image capture unit 108 has a predetermined distance from the second image capture unit 110, which distance defines a stereo base of the stereo endoscope. The first and second image capture units 108, 110 are preferably each a camera. The evaluation device 106 is preferably configured to receive image data in the form of measurement images from the first and second image capture units 108, 110 and to evaluate them. Particularly preferably, the evaluation device 106 comprises at least one processor (not shown in more detail) for image processing. It is understood that, in other embodiments, the evaluation device 106 can preferably be arranged outside the image capture device 104. The evaluation device 106 is preferably designed as a so-called camera control unit (CCU). Preferably, at least a preprocessing of the captured measurement images 109, 111 can take place in the image capture device 104.

A lens assembly 114 is assigned to the first and second image capture units 108, 110. The lens assembly 114 comprises, for example, a cover glass and optical units 116, 118 with apertures that are assigned to the image capture units 108, 110. The optical units 116, 118 define the respective field of view of the image capture units 108, 110. Each of the two image capture units 108, 110 is assigned to an observation channel 120, 122. The observation channels 120, 122 are each designed to transmit the measurement images in the form of signal-like image information to the evaluation device 106 or to the at least one processor. For providing the image information, each of the image capture units 108, 110 is assigned a signal converter 124, 126. The signal converters 124, 126 are each configured to convert the optically captured measurement images into image information. For example, the signal converters 124, 126 are photo chips.

The first image capture unit 108 is configured to capture at least a first measurement image of at least one movable measurement object 112. The measurement object 112 is shown here by way of example as a letter P. However, the measurement object 112 is normally preferably a human or animal organ or another part of a human or animal body or a component. The first image capture unit preferably captures the at least one measurement image of the measurement object 112 from a first viewing angle.

The second image capture unit 110 is configured to capture at least a second measurement image of the at least one movable measurement object 112. The first and second image capture units 108, 110 are each configured to capture the first and second measurement images, preferably in a temporally synchronized manner. A first and a second measurement image thus captured preferably form a stereo image pair.

The evaluation device 106 is designed to ascertain stereo measurement image information from the stereo image pair or from the signal-based image information of the first and second measurement images by means of known stereo reconstruction methods. In the stereo measurement image information, depth information is available for each captured image point of the at least one measurement object 112, which depth information can, for example, be used to calculate a distance between two measurement points on the measurement object 112 in Euclidean space.

The stereo measurement image information can preferably be transmitted via a first and/or a second output channel 128, 130 to a user output device 132, which provides and preferably graphically displays the stereo measurement image information to a user as a first (stereo) measurement image 134. The user output device 132 may, for example, be a display. The at least one measurement object 112 is displayed in the form of an observation object 136 on the user output device 132. A user can preferably displace a cursor 138 relative to the observation object 136 or to the virtualized measurement object 112 by means of a user input device (not shown), for example in order to determine a measurement point in relation to the measurement object.

It is understood that the measuring device 100 is configured to basically capture or provide a plurality of measurement images of the measurement object and thus a plurality of stereo measurement images for each stereo image pair. Particularly preferably, it is possible with the measuring device 100 to record a (live) video of the at least one measurement object 112, which (live) video is composed of a plurality of individual image pairs that are captured one after the other with a predefined time interval (determined by the frame rate).

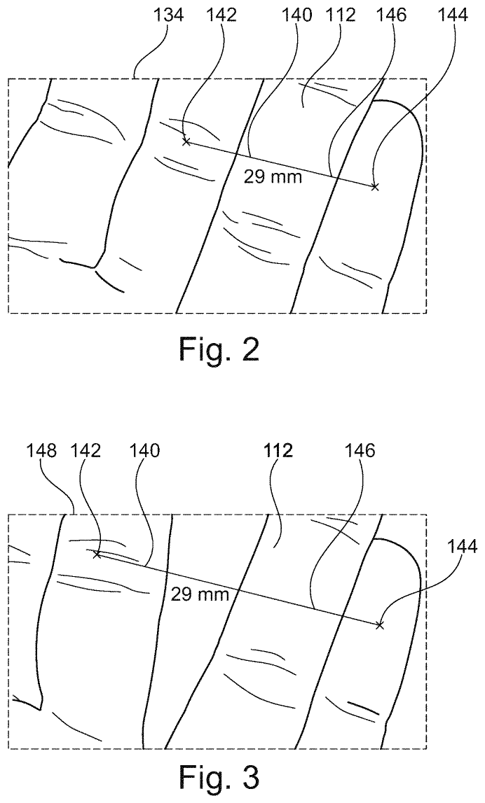

FIG. 2 shows, by way of example, a first stereo measurement image 134 in simplified two-dimensional form, which was recorded, for example, by the measuring device 100 according to the disclosure. It is evident that the measurement object is now no longer the measurement object shown by way of example in FIG. 1, but a human body part is represented as the measurement object 112 by the first stereo measurement image 134. The at least one measurement object 112 is represented here by fingers of a hand. However, the at least one measurement object 112 may also comprise any other type of measurement object, for example a part of an anatomy and/or one or more organs of the human or animal body. Alternatively, the at least one measurement object 112 may also be a component. According to FIG. 2, in the first stereo measurement image 134, a distance 140 between a first measurement point 142 and a second measurement point 144 is represented by way of example, which distance is displayed to the user visually both as a measuring section 146 and additionally as a numerical value, in the example 29 mm, as graphic elements superimposed on the first stereo measurement image 134.

The first measurement point 142 is determined and/or set in relation to the at least one movable measurement object 112, for example in the first stereo measurement image 134, by a user, preferably by means of the cursor 138 displayed on the user output device 132 and/or in the user's glasses. The second measurement point 144 is determined and/or set in relation to the at least one movable measurement object 112, for example in the first stereo measurement image 134, by a user, preferably by means of the cursor 138 displayed on the user output device 132 and/or in the user's glasses.

FIG. 3 shows, by way of example, a second (stereo) measurement image 148 following the first stereo measurement image 134 in time. The at least one measurement object 112 is located in a second position according to the second stereo measurement image 148. The position change of the at least one measurement object 112 is due to a movement of the measurement object 112, in the present example case due to a rhythmic and/or frequent opening and closing of the fingers of the hand. The first and second stereo measurement images 134, 148 each represent an exemplary snapshot of this rhythmic or frequent movement.

The first and second stereo measurement images 134, 148 here are each individual images of a plurality of temporally successive measurement images (cf. explanations for FIG. 1), which, when placed one after the other, result in a video recording of the at least one measurement object 112. Each stereo measurement image of the plurality of stereo measurement images, i.e., also the first and second stereo measurement images 134, 148, results in each case from a stereo image pair which is reconstructed by known stereo reconstruction methods so that depth information is preferably available for each captured image point of the at least one measurement object 112. The distance 140 between the first and second measurement points 142, 144 in Euclidean space is calculated from each stereo image pair and/or from the already stereo-reconstructed stereo measurement image.

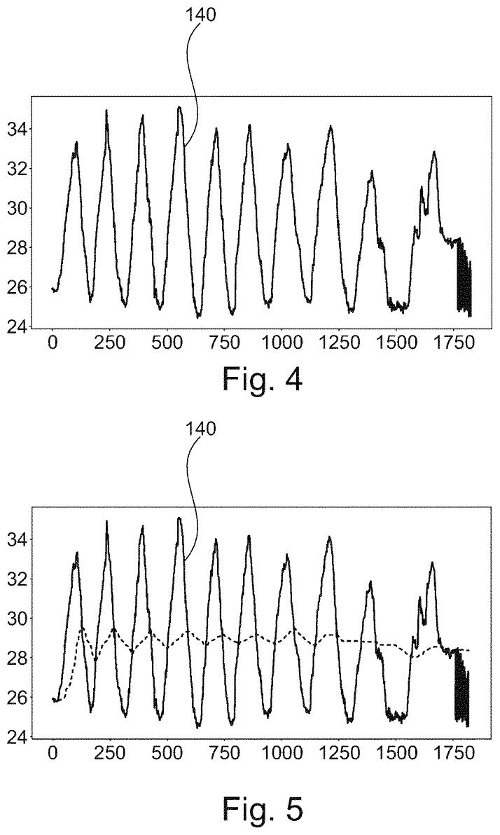

This results in a time series of measuring sections 146, which is shown by way of example in the diagram shown in FIG. 4. The image sequence number (frame number) is shown on the abscissa. The ordinate shows the changing distance in millimeters of the opening and closing fingers. The change in distance between two measurement points on the movable measurement object 112, which are visually marked by the two measurement points 142, 144, can be calculated independently of the optical or visualized adjustment of the measurement points 142, 144 (their adhesion to the measurement object) by methods for determining the optical flow and by similar methods.

As can be seen from a combination of FIGS. 2 and 3, the first and second measurement points 142, 144 are corrected according to the method according to the disclosure with regard to their position depending on the movement of the at least one measurement object 112 and thus adhere visually to the measurement object 112 in the view displayed to the user of the measuring device 100. The open-source function of openCV, cv2.calcOpticalFlowPyrLK( ) was used for this purpose. In principle, however, other optical flow calculation methods and/or landmark-based tracking for the position tracking and position correction of the two measurement points 142, 144 are also possible.

However, according to the exemplary embodiment shown in FIGS. 2 and 3, the measuring section 146 displayed to the user is not evaluated statistically according to the method likewise according to the disclosure. Rather, the distance 140 between the two measurement points 142, 144 is merely averaged over time. As shown in FIG. 3, this temporal average value image results in an incorrect numerical value, here 29 mm by way of example, being displayed to the user in the superimposed graphic element for the distance 140 currently represented in the second stereo measurement image 148. This erroneous representation can be fundamentally improved by the method according to the disclosure according to the second aspect.

In accordance with the erroneous representation of the distance 140 in the second stereo measurement image 148 shown in FIG. 3, the distance image sequence number known from FIG. 4 is shown in FIG. 5 together with an average value curve calculated for it. It is evident that the resulting average over time is a distance 140 with an average value of approximately 29 mm. However, due to averaging the distance 140 over time, the displayed numerical value lags behind in time, as shown by way of example in FIG. 3, which can lead to uncertainty about the correctness of the display. The statistical error of this measurement is 3 mm in the experiment and is therefore inaccurate in comparison to the evaluation made possible according to the disclosure.

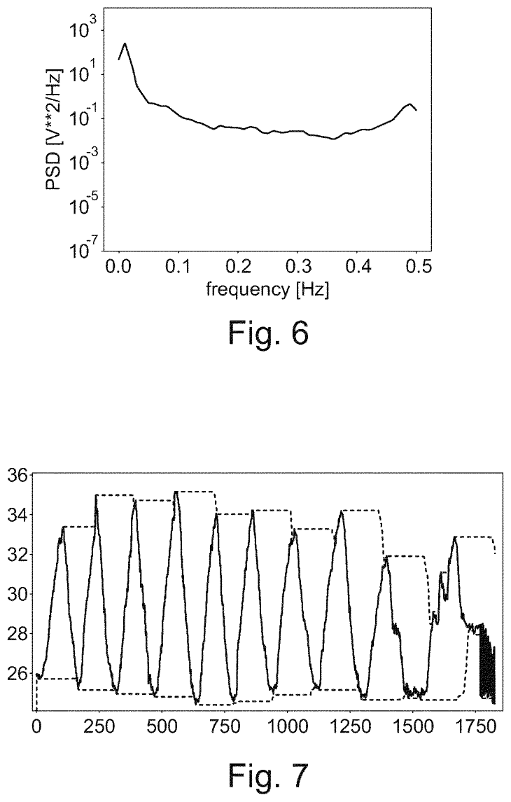

Instead of the averaging shown by way of example in FIG. 5, the ascertained time series of the measuring section 146 is additionally analyzed according to the disclosure by means of statistical methods, for example by calculating a periodicity and/or an amplitude. The periodicity and/or an amplitude represent, by way of example, the at least one statistical characteristic variable. By way of example, a periodogram (max. at 0.006 1/frames corresponds to 170 frames) of the frequency analysis is shown in FIG. 6 as the result of such an analysis. In addition, an amplitude (displayed as minima and maxima) of the ascertained time series of the measuring section 146 was ascertained by way example and is shown in FIG. 7. By calculating these statistical parameters, minimum and maximum values as well as, if applicable, frequencies of a movement of the at least one measurement object can be displayed to the user, preferably graphically.

Particularly preferably, at least one vital parameter that is significant for the at least one measurement object can be ascertained from the statistically analyzed movement of the at least one measurement object 112. If the at least one measurement object 112 is, for example, a human or animal heart, the method according to the disclosure makes it possible to ascertain a respiratory rate and/or a heart rate on the basis of the at least one statistical characteristic variable. If the at least one measurement object 112 comprises at least one vein and/or artery situated at the surface of the object, it is possible to ascertain a heartbeat on the basis of the at least one statistical characteristic variable by means of the method according to the disclosure.

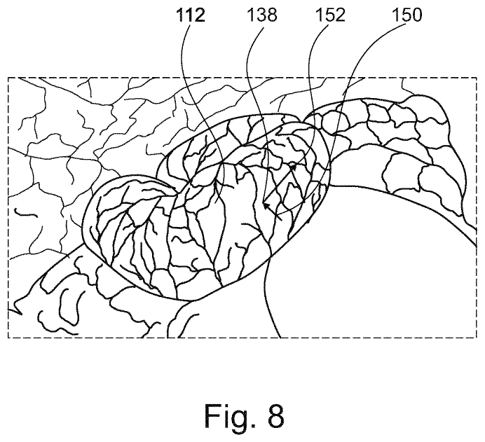

FIG. 8 shows an application example of the method according to the disclosure according to the first aspect. In order to determine the position of the cursor 138 for selecting measurement points 142, 144, two different displacement vectors are calculated. On the one hand, a position displacement vector 150 between two consecutive stereo measurement images is determined from optical tracking as described above. In addition, a movement vector 152 is determined from a, preferably manual, user input, e.g., by means of a joystick, a keyboard or a mouse. The two vectors 150, 152 are superposed and the cursor 138 can be displaced accordingly. This results in a cursor position only changing relative to the at least one measurement object 112. The cursor 138 or measurement point thus preferably adheres to the measurement object as long as the user does not specify a different movement or perform a different input.

1. Method for correcting a position of a measurement point in a measurement image, in particular in an endoscopic and/or exoscopic and/or microscopic measurement image, comprising at least the steps of:
Capturing a first measurement image of at least one movable measurement object;
Determining the at least one measurement point in relation to the at least one movable measurement object in the first measurement image;
Capturing at least one second measurement image, following the first measurement image in time, of the at least one movable measurement object;
Calculating a position displacement vector between the at least one measurement point in the first measurement image and an image point, corresponding to the at least one measurement point, in the second measurement image; and
Correcting a position of the at least one measurement point determined in relation to the first measurement image, in the second measurement image on the basis of the calculated position displacement vector.

2. Method according to claim 1, characterized in that a position change between the at least one measurement point determined in relation to the first measurement image and the image point corresponding thereto in the second measurement image is based on a movement of the movable measurement object.

3. Method according to claim 1, characterized in that calculating the position displacement vector takes place on the basis of an optical flow algorithm and/or an elastic image registration algorithm and/or a point cloud registration algorithm and/or landmark-based tracking.

4. Method according to claim 1, characterized in that the first and second measurement images are each an individual image of a stereo video sequence.

5. Method according to claim 1, characterized in that determining the at least one measurement point in relation to the at least one movable measurement object takes place on the basis of a user input, preferably by moving a cursor within the first measurement image.

6. Method according to claim 5, characterized in that correcting the position of the at least one measurement point includes superimposing a movement vector of the cursor for determining the measurement point with the position displacement vector.

7. Method according to claim 1, characterized in that the at least one measurement point is represented, in particular always, in its corrected position on a user output device.

8. Method for outputting at least one statistical characteristic variable for a movable measurement object, which is in particular comprised in an endoscopic and/or exoscopic and/or microscopic measurement image, wherein the method comprises at least the steps of:
Providing a plurality of temporally successive measurement images of the at least one movable measurement object, wherein the plurality of measurement images comprises at least one measuring section which is to be output in relation to the movable measurement object and is calculated between a first measurement point, which is determined in relation to the at least one movable measurement object, and a second measurement point, which is determined in relation to the at least one movable measurement object;
Ascertaining, by statistical evaluation, the at least one statistical characteristic variable in the plurality of temporally successive measurement images, which statistical characteristic variable is at least partially related to a periodic and/or frequency-based movement of the at least one movable measurement object;
Outputting the at least one statistical characteristic variable, which describes the movement of the at least one movable measurement object at least partially.

9. Method according to claim 8, characterized in that the at least one statistical characteristic variable comprises at least one of a frequency, an amplitude, a minimum value, a maximum value, an average value, a standard deviation, and a statistical error indicator.

10. Method according to claim 8, characterized in that an average resulting measurement error is output for the at least one statistical characteristic variable.

11. Method according to claim 8, characterized in that a position change of the first measurement point and/or of the second measurement point in the plurality of temporally successive measurement images, which position change is caused at least partially by a movement of the at least one movable measurement object, is corrected by: the method according to
Capturing a first measurement image of at least one movable measurement object;
Determining the at least one measurement point in relation to the at least one movable measurement object in the first measurement image;
Capturing at least one second measurement image, following the first measurement image in time, of the at least one movable measurement object;
Calculating a position displacement vector between the at least one measurement point in the first measurement image and an image point, corresponding to the at least one measurement point, in the second measurement image; and
Correcting a position of the at least one measurement point determined in relation to the first measurement image, in the second measurement image on the basis of the calculated position displacement vector.

12. Method according to claim 1, characterized in that the first measurement image and/or the second measurement image and/or the plurality of measurement images are processed by stereo reconstruction of a respective stereo image pair on the basis of optical and/or dimensional parameters of the image capture device.

13. Measuring device comprising:
an image capture device with at least a first and a second image capture unit spaced apart from the first one, an evaluation device, and
a processor configured to at least partially perform the steps of the method according to claim 1 and/or of the method according to claim 8.

14. Measuring device according to claim 13, characterized in that the measuring device comprises a stereo endoscope or a stereo exoscope or a stereo microscope or a laryngoscope.

15. Measuring device according to claim 13, characterized in that it comprises a user input device and/or a user output device.
